(19) United States
(12) Patent Application Publication
(10) Pub. No.: US 2003/0077342 A1
(43) Pub. Date: Apr. 24, 2003

(54) Anti-inflammatory complex containing eicosapentaenoic acid

(75) Inventor: Philip B. Maffetone, St. Augustine, FL (US)

Correspondence Address: STEPTOE & JOHNSON LLP, 1330 Connecticut Avenue, N.W., Washington, DC, 20036, US

(73) Assignee: MAF Group, LLC, Boonton, NJ (US)

(21) Appl. No.: 10/291,694

(22) Filed: Nov. 12, 2002

Related U.S. Application Data
(63) Continuation of application No. 09/971,654, filed on Oct. 9, 2001, now abandoned.

(51) Int. Cl. A61K 35/78; A61K 31/202

(52) U.S. Cl. 424/754; 424/736; 424/756; 424/776; 514/560

(57) ABSTRACT

A nutritional supplement includes eicosapentaenoic acid (EPA), docosahexaenoic acid (DHA), ginger, sesame, citrus, garlic and tumeric. The nutritional supplement preferably includes about 170 to 200 mg of EPA, about 113 to 133 mg of DHA, about 50 to 100 mg of ginger, about 50 to 100 mg of sesame, about 50 to 100 mg of citrus, about 50 to 100 of garlic, and about 10 to 30 mg of tumeric. The nutritional supplement may be used in a method of treating inflammation by administering to a patient in need thereof an effective dose of the nutritional supplement.

### BACKGROUND OF THE INVENTION

1. Field of the Invention

The present invention relates to a nutritional supplement containing a variety of naturally occurring substances useful for treating inflammatory conditions.

2. Description of the Related Art

Fish oil contains the omega-3 fatty acids eicosapentaenoic acid (EPA) and docosahexaenoic acid (DHA). Most fish oil supplements are 18% EPA and 12% DHA (30% omega-3 fatty acids). These omega-3 fatty acids help regulate blood triglycerides, may inhibit the progression of atherosclerosis, influence blood clotting, and are important for nervous system development in the fetus and newborn and brain function in adults. EPA and DHA also have anti-inflammatory activity. As a result, fish oil is used to help people with various inflammatory conditions, and chronic inflammatory states that may be either symptomatic or asymptomatic.

Fish oil may help people with a wide variety of disorders, including the following: some types of cancers, heart disease, immune problems, pain syndromes, diabetes mellitus, epilepsy, some types of brain disorders including Alzheimer, Schizophrenia and depression, some intestinal diseases including Crohn's disease and ulcerative colitis, hyperlipidemia, arthritis, some types of skin conditions, some types of kidney disease, and others including positively influencing gene expression.

EPA and DHA are found in a variety of animal sources including mackerel, salmon, herring, sardines, sable fish (black cod), anchovies, albacore tuna, and wild game.

Fish oil is easily damaged by oxidation. Accordingly, a few milligrams or IUs of vitamin E are included in fish oil supplements to serve as an anti-oxidant. Doctors often recommend that people who supplement with fish oil or DHA take vitamin E supplements to protect EPA and DHA within the body from oxidative damage.

There are a number of known EPA-DHA complexes used as a totally natural source of the omega-3 essential fatty acids, EPA and DHA, concentrated from cold water fish oil. Each capsule typically provides:

| | | |
|---|---|---|
| EPA (Eicosapentaenoic Acid) | 180-400 | mg |
| DHA (Docosahexaenoic Acid) | 120-600 | mg |
| Natural Marine Lipid Concentrate | 1-2 | g |
| Vitamin E (D-Alpha Tocopherol) | 10 | IU. |

A typical recommended dosage is 1 to 6 capsules daily.

There is a need in the art for a new and improved nutritional supplement containing EPA. There is also a need in the art for a nutritional supplement containing EPA with improved anti-inflammatory properties.

An object of the present invention is to provide a new and improved nutritional supplement containing EPA. Another object of the present invention is to provide a nutritional supplement containing EPA with improved anti-inflammatory properties. These and other objects of the present invention will become more apparent from a consideration of the following description and claims.

### BRIEF SUMMARY OF THE INVENTION

The nutritional supplement of the present invention includes EPA, DHA, ginger, sesame, citrus, garlic and tumeric as anti-inflammatory ingredients. The nutritional supplement preferably includes about 170 to 200 mg of EPA, about 113 to 133 mg of DHA, about 50 to 100 mg of ginger, about 50 to 100 mg of sesame, about 50 to 100 mg of citrus, about 50 to 100 mg of garlic, and about 10 to 30 mg of tumeric. The ginger is preferably ginger root, the sesame is preferably sesame seed oil, the citrus is preferably citrus skin oil, and the garlic is preferably garlic powder (odorless).

The nutritional supplement of the present invention more preferably includes:

| | | |
|---|---|---|
| EPA | 175 to 185 | mg |
| DHA | 117 to 123 | mg |
| Ginger | 70 to 80 | mg |
| Sesame | 70 to 80 | mg |
| Citrus | 70 to 80 | mg |
| Garlic | 70 to 80 | mg |
| Tumeric | 10 to 30 | mg. |

The nutritional supplement of the present invention most preferably contains:

| | | |
|---|---|---|
| Organic EPA | 180 | mg |
| Organic DHA | 120 | mg |
| Organic ginger root | 75 | mg |
| Organic sesame seed oil | 75 | mg |
| Organic citrus skin oil | 75 | mg |
| Organic garlic powder (odorless) | 75 | mg |
| Organic tumeric | 20 | mg. |

The anti-inflammatory complex containing EPA is a natural remedy, especially for the relief of chronic inflammation. Chronic inflammation is often an asymptomatic condition that may be the first stage of a variety of chronic diseases, including cancer, heart disease, neurodegenerative disorders (e.g., Alzheimer), and others.

Accordingly, the present invention also relates to a method of treating inflammation which includes administering to a patient in need thereof a nutritional supplement including EPA, DHA, ginger, sesame, citrus, garlic and tumeric. The preferred mode of administration is by capsule or the like. The recommended dosage is 1 to 16 capsules per day.

While the preferred use of the present invention is for inflammatory conditions, it will be understood that the present invention may be used as a nutritional supplement for any of the conditions EPA, DHA, ginger, sesame seed oil, citrus skin oil, garlic oil or tumeric are known to be used for in the art.

### DETAILED DESCRIPTION OF THE INVENTION

In accordance with the present invention, a nutritional supplement is provided which includes EPA, DHA, ginger, sesame, citrus, garlic and tumeric.

“EPA” refers to the fatty acid eicosapentaenoic acid. The nutritional supplement preferably contains about 170 to 200 mg, more preferably about 175 to 185 mg, of EPA. The nutritional supplement most preferably contains about 180 mg of EPA.

“DHA” refers to the fatty acid docosahexaenoic acid. The nutritional supplement of the present invention preferably contains about 113 to 133 mg, more preferably about 117 to 123 mg, of DHA. The nutritional supplement most preferably contains about 120 mg of DHA.

The ratio of EPA:DHA in the nutritional supplement is preferably about 1.5:1.

One naturally occurring source of EPA and DHA is fish oil. Fatty fish such as salmon, mackerel, sardines, ocean trout and herring contain the highest amounts of EPA/DHA. Other sources of EPA/DHA include fin fish such as whiting and flounder, plants such as seaweeds, and microorganisms.

Ginger is a perennial plant that grows in the United States, India, China, Mexico, and several other countries. The dried rhizome (underground stem or root) of ginger contains approximately 1-4% volatile oils. These are the medically active constituents of ginger, and they are also responsible for ginger's characteristic odor and taste. The aromatic principles include zingiberene and bisabolene, while the pungent principles are known as gingerols and shogaols.

Ginger is a classic tonic for the digestive tract, may be helpful for motion sickness, has anti-fungal and anti-oxidant effects, and may protect the stomach from the damaging effect of non-steroidal anti-inflammatory drugs. Ginger also has anti-inflammatory properties and has been used for the treatment of certain cancers, cardiovascular disease, thrombosis, arteriolsclerosis, atherosclerosis, hyperlipoproteinemia, and other conditions. The nutritional supplement of the present invention preferably contains about 50 to 100 mg, more preferably about 70 to 80 mg, and most preferably about 75 mg of ginger.

Sesame seed oil is derived from a plant species called Sesamum indicum, which is a herbaceous annual belonging to the Pedaliaceae family. Sesame seed oil, which contains the lignan sesamin, has been used for a variety of therapeutic purposes. The oil is also used in certain cosmetics and skin softening products.

Sesame oil, which is also referred to as benne, gingili, or teel oil, is made from the black seeds of Sesamum indicum. Sesame seed oil has been shown to have anti-inflammatory, anti-oxidant, and anti-hypertensive properties, and as such is believed to have several other important properties, including anti-cancer and antibacterial effects. Sesame seed oil can also increase DHA levels in the body. The nutritional supplement of the present invention preferably contains about 50 to 100 mg, more preferably about 70 to 80 mg, and most preferably about 75 mg of sesame seed oil.

Citrus skin oil can be prepared from a variety of citrus fruits, preferably oranges. Citrus skin oils contain significant levels of monoterpenes, including limonene, which have anti-inflammatory and anti-oxidant effects, with studies supporting their anti-cancer effects, especially with cancers of the breast, colon and skin. The nutritional supplement of the present invention preferably contains about 50 to 100 mg, more preferably about 70 to 80 mg, and most preferably about 75 mg of citrus skin oil.

Garlic is closely related to onion and chives. The sulfur compound allicin, produced by crushing or chewing fresh garlic, in turn produces other sulfur compounds: ajoene, allyl sulfides, and vinyldithiins. Research suggests that garlic powder may have anti-inflammatory actions, along with antimicrobial, antithrombotic, antitumor, hypolipidaemic, and hypoglycemic effects. The nutritional supplement of the present invention preferably contains about 50 to 100 mg, more preferably about 70 to 80 mg, and most preferably about 75 mg of garlic powder (odorless).

Tumeric is the common name for Curcuma domestica Loir or Curcuma longa L. Tumeric is a perennial herb. The rhizomes are robust, fleshy and branch at right angles forming a dense clump. The rhizomes are aromatic, orange-brown in color on the outside and bright orange in the inside. The tubers are elliptical in shape, fleshy, white in color and grow vertically downwards. The rhizomes are the plant parts used. Tumeric, which contains a substance called curcumin, has both anti-inflammatory and anti-oxidant effects. As such, it has been shown to have anti-cancer effects. The nutritional supplement of the present invention preferably contains about 10 to 30 mg, most preferably about 20 mg of tumeric.

The nutritional supplement is preferably provided as a capsule and includes a liquid or dry inner filling of active ingredients and an outer shell. In the preferred embodiment, the inner filling is a liquid mixture which is contained within a gelatin capsule, such as a “softgel” type capsule. The inner filling includes EPA, DHA, ginger, sesame, citrus, garlic and tumeric.

The components of the inner filling are preferably solubilized in a liquid to form a liquid mixture. Suitable liquids include vegetable oils such as sesame oil. The inner filling can contain other components, including fillers, and components useful for adjusting the isotonic properties of the filling (such as glycerin or beeswax).

When the inner filling of the nutritional supplement of the present invention is a liquid, the outer shell is preferably composed of a gelatin as well as other optional components such as glycerin and a coloring agent (carob). Such a nutritional supplement can be obtained by filling the outer shell with the premixed inner filling.

The recommended dosage is 1 to 16 capsules per day.

While the foregoing invention has been described in some detail for purposes of clarity and understanding, it will be appreciated by one skilled in the art from a reading of this disclosure that various changes in form and detail can be made without departing from the true scope of the invention and appended claims. For example, the nutritional supplement may contain other ingredients such as about 1 IU of Vitamin E to protect EPA and DHA within the body from oxidative damage without departing from the true scope of the invention and appended claims.

I claim:

1. A nutritional supplement comprising eicosapentaenoic acid (EPA), docosahexaenoic acid (DHA), ginger, sesame, citrus, garlic and tumeric.

2. The nutritional supplement of claim 1, comprising about 170 to 200 mg of EPA, about 113 to 133 mg of DHA, about 50 to 100 mg of ginger, about 50 to 100 mg of sesame, about 50 to 100 mg of citrus, about 50 to 100 mg of garlic, and about 10 to 30 mg of tumeric.

3. The nutritional supplement of claim 1, wherein the ginger is organic ginger root.

4. The nutritional supplement of claim 1, wherein the sesame is organic sesame seed oil.

5. The nutritional supplement of claim 1, wherein the citrus is organic citrus skin oil.

6. The nutritional supplement of claim 1, wherein the garlic is garlic powder.

7. The nutritional supplement of claim 1, wherein the tumeric is organic tumeric.

8. A method of treating inflammation, comprising administering to a patient in need thereof an effective amount of a nutritional supplement comprising eicosapentaenoic acid (EPA), docosahexaenoic acid (DHA), ginger, sesame, citrus, garlic and tumeric.

9. The method of claim 8, wherein the nutritional supplement comprises about 170 to 200 mg of EPA, about 113 to 133 mg of DHA, about 50 to 100 mg of ginger, about 50 to 100 mg of sesame, about 50 to 100 mg of citrus, about 50 to 100 mg of garlic, and about 10 to 30 mg of tumeric.

10. The method of claim 8, wherein the ginger is organic ginger root.

11. The method of claim 8, wherein the sesame is organic sesame seed oil.

12. The method of claim 8, wherein the citrus is organic citrus skin oil.

13. The method of claim 8, wherein the garlic is garlic powder.

14. The method of claim 8, wherein the tumeric is organic tumeric.

15. A nutritional supplement comprising:

| | | |
|---|---|---|
| Eicosapentaenoic acid | 175 to 185 | mg |
| Docosahexaenoic acid | 117 to 123 | mg |
| Ginger | 70 to 80 | mg |
| Sesame | 70 to 80 | mg |
| Citrus | 70 to 80 | mg |
| Garlic | 70 to 80 | mg |
| Tumeric | 10 to 30 | mg. |

16. An organic nutritional supplement comprising:

| | | |
|---|---|---|
| Eicosapentaenoic acid | 180 | mg |
| Docosahexaenoic acid | 120 | mg |
| Ginger root | 75 | mg |
| Sesame seed oil | 75 | mg |
| Citrus skin oil | 75 | mg |
| Garlic powder | 75 | mg |
| Tumeric | 20 | mg. |

17. A method of treating inflammation, comprising administering to a patient in need thereof an effective amount of a nutritional supplement comprising:

| | | |
|---|---|---|
| Eicosapentaenoic acid | 175 to 185 | mg |
| Docosahexaenoic acid | 117 to 123 | mg |
| Ginger | 70 to 80 | mg |
| Sesame | 70 to 80 | mg |
| Citrus | 70 to 80 | mg |
| Garlic | 70 to 80 | mg |
| Tumeric | 10 to 30 | mg. |

18. A method for treating inflammation, comprising administering to a patient in need thereof an effective amount of an organic nutritional supplement comprising:

| | | |
|---|---|---|
| Eicosapentaenoic acid | 180 | mg |
| Docosahexaenoic acid | 120 | mg |
| Ginger root | 75 | mg |
| Sesame seed oil | 75 | mg |
| Citrus skin oil | 75 | mg |
| Garlic powder | 75 | mg |
| Tumeric | 20 | mg. |
